(19) United States
(12) Patent Application Publication
(10) Pub. No.: US 2011/0174733 A1
(43) Pub. Date: Jul. 21, 2011

(54) FILTER SYSTEM FOR EXTRACORPOREAL DEPLETION OF ACTIVATED POLYMORPHONUCLEAR LEUKOCYTES (PMNs)

(76) Inventor: Hans-Werner Heinrich, Greifswald (DE)

(21) Appl. No.: 13/061,181

(22) PCT Filed: Aug. 27, 2009
(86) PCT No.: PCT/DE2009/001209

§ 371 Date: Apr. 8, 2011

(30) Foreign Application Priority Data
Sep. 1, 2008 (DE) 10 2008 045 127.4

(51) Int. Cl. A61M 1/16; B01D 61/00; B23P 17/04

(52) U.S. Cl. 210/645; 210/321.62; 29/592

(57) ABSTRACT

The invention relates to a filter system for removing activated PMNs and mediators which are responsible for the activation thereof, and optionally mediators which are simultaneously, but independently of activated PMNs, responsible for a state of illness from body fluids, process for production thereof and use thereof. The inventive filter system for continuous removal of activated PMNs from blood consists of molecules which recognize and bind activated PMNs, molecules which recognize and bind PMN-activating mediators, molecules which recognize and bind C3a and/or C5a, biocompatible surfaces (carriers) on which the molecules a., b. and c. are bound covalently or adsorptively, a casing for accommodating the active carriers, valves and tubes. The field of use of the invention is medicine.

### CROSS-REFERENCE TO RELATED APPLICATIONS

This application is the U.S. National Phase of PCT International Patent Application No. PCT/DE2009/001209, filed on Aug. 27, 2009, and claiming priority to German application no. 10 2008 045 127.4, filed on Sep. 1, 2008. Both of those applications are incorporated by reference as if fully rewritten herein.

### BACKGROUND OF THE INVENTION

1. Field of the Invention

Embodiments relate to a filter system for removal of activated PMNs and mediators from body fluids, which are responsible for their activating, as well as mediators when indicated, which are responsible for the pathogenic situation at the same time but non-related to activated PMNs. Embodiments further relate to a procedure for its manufacturing and use.

2. Background of the Related Art

During the last years, the knowledge regarding the patho-physiology of the systemic inflammation (Systemic Inflammatory Response Syndrome—SIRS) and multiple organ failure has significantly improved. Obviously, cytokines play a decisive role in the regulation as well as de-regulation of systemic inflammation. Activated CD14⁺ cells produce cytokines (TNFα, Interleukin 1 (IL-1), IL-6, IL-8), which influence their target cells via cytokine-receptors.

Besides cytokines, immune competent lymphocytes and PMNs have a causative function in the defense of infections and maintenance of the organ integrity after lesion/surgery.

The complement system will be activated parallel to the stimulation of monocytes, macrophages, lymphocytes and PMNs. It is an integrated component of the immunologic defense in mammals for the immediate and unspecific fight of bacterial microorganisms and foreign particles. From all the complement proteins which can be found in the blood serum, and which are mostly pro-enzymes activated by proteolytic cleavage, the C3-protein plays a central role with its serum concentration of 1g/L. After contact of the microorganisms with C3, the complement protein C3a gets cleaved off. The resulting C3b induces on the one hand the generating of the C5-convertase (the alternative pathway of complement activation), and on the other hand the reaction will be amplified by serum factors which dock on C3b and so form the C3-convertase. Because the increasing amount of C5-convertase and the presence in the serum of C5; C5 will be gradually more protoelyticly cleaved under generation of C5a. Additional complement factors (C6-C9) dock on the C5b until finally the polymer hydrophobe membrane attack complex (MAC) is generated, which integrates in the bacterial membrane and forms pores leading to phagocytosis and the elimination of the microorganisms (and the attached MAC). The complement factors C3a and C5a (anaphylatoxins), which are liberated In the process of complement activating, cause the attraction of phagocyting cells at the site of the bacterial intrusion by increasing the blood vessel permeability and by the induced release of chemotoxins. The reduction of the number of bacterial causes a decrease of complement activation. This unspecific and immediate reaction is tightly interwoven with other immunologic defense systems, so e.g. regulate complement factors the synthesis and excretion of cytokines which are essential for the cellular defense system. C3a and C5a are attached to specific cell-bound receptors to facilitate the inflammatory activity. The receptors are differently strongly expressed depending on the immune reactivity. To maintain the immune defense permanently ready to act, activated complement factors are an integrated part in serum of healthy people with a concentration of 1-10 ng/ml, and not only detectable after an intrusion of microorganisms.

Plasma levels of anaphylatoxins can be increased to about more than 1,000 times especially during developed septicemia, acute lung failure and moribund patients.

The local increase of complement can be connected with harmful consequences for the tissue, as after heart attack, cerebral malaria, brain trauma, and ischemic condition of other origin.

PMNs and immune competent lymphocytes are essential cellular components for a successful immediate and late reaction in the defense of infections and maintaining the organ integrity after lesion/surgery.

An increased apoptosis of lymphocytes in the late stage of a generalized inflammation is a surviving-chance reducing complication. Hotchkiss et al. (Accelerated lymphocyte death in sepsis occurs by both the death receptor and mitochondrial pathways. J Immunol 174: 5110-5118, 2005) demonstrate that apoptosis of the immune competent lymphocytes during the hypo-inflammatory stage plays an additional role in the pathogenesis of sepsis. A very high apoptosis rate of lymphocytes could be measured in patients who died due to sepsis, whereas the degree of lymphocyte apoptosis correlates with the severity of the sepsis. The leucopenia has its cause in the increased apoptosis especially of CD4⁺ T-cells and B-cells. Based on this lymphopenia the immune system is not anymore in the position to react adequately to the pathogen. PMNs and the complement system form together the first barrier of the innate immune system. PMNs get activated at the place of lesion and/or place of intrusion of pathogens as result of stimulation by humeral pro-inflammatory mediators (TNFα, IL-1β, IL-6, IL-8, GM-CSF, and interferon-gamma). Those cytokines are synthesized by different cell types, including immune competent cells. An excessive freeing of those cytokines results into SIRS, which finally leads to multi-organ failure.

The activated PMNs internalize the microorganisms and release cytotoxic substances like enzymes and activated oxygen (H2O2). The life span of the PMNs is very short, 6-8 hours. The aging PMNs underlie the apoptosis and are finally internalized and catabolized by macrophages. The same process also takes place during locale activation. The activated PMNs are an essential part of the local inflammation. When the problem is solved, the activated PMNs underlie the apoptosis and are consumed by macrophages without releasing the cytotoxic substances which have been accumulated in their insides. With the declining of the local inflammation, mediators cause counteraction to activation and the phagocytosis. That is why the non-selective cytotoxicity cannot cause harm in the surrounding tissue.

The delayed initiation of apoptosis in activated PMNs is also a wanted physiological process to increase the PMN-function in reaction to infection or lesion. Persisting pro-inflammatory hypercytokinemia and the start of a systemic inflammatory reaction as the result of, e.g. an excessive injury leads to the obstruction of the apoptotic clearance of the activated PMNs.

Those PMNs which do not go into apoptosis can be used from the organism to fight the infection. But the wanted effect of an increased infection defense also generates the risk that cytotoxic substances of the activated PMNs are released in the surrounding tissue.

Delayed PMN-apoptosis could be measured in the blood of patients suffering from SIRS, sepsis, ARDS, trauma or burning. Hirano et al. (Modulation of Polymorphonuclear Leukocyte Apoptosis in the Critically III by Removal of Cytokines with Continuous Hemodiafiltration, Blood Purif 2004; 22:188-197) demonstrated a significant correlation between high IL-6-level and delayed apoptosis. The severity of the systemic inflammation (APACE II scores) correlates with the peak of the IL-6 level.

The severity of the systemic inflammation is linked to disorders in the PNS function. Tavares-Murta et al. (Failure of neutrophil chemotactic function in septic patients. Crit Care Med 30:1056-1061, 2002) describe a reduced chemotaxis of PMNs from sepsis patients. Stephan F et al. (Impairment of polymorphonuclear neutrophil functions precedes nosocomial infections in critically ill patients. Crit Care Med 30:315-322, 2002) found a reduced phagocytosis capacity in PMNs from sepsis patients, and also Holzer et al. (Phagocytosis by emigrated, intra-abdominal neutrophils is depressed during human secondary peritonitis. Eur Surg Res 34:275-284, 2002.) observed a long lasting reduction of phagocytosis capacity in PMNs from peritonitis patients.

This “PMNs dysfunction syndrome” is combined with an increased production of free cytotoxic oxygen and H₂O₂ (Wittmann et al.: Differential effects of clindamycin on neutrophils of healthy donors and septic patients. Int Immunopharmacol 4:929-937, 2004).

If and in what direction a PMN dysfunction will develop, cannot be predicted until now. It can lead to an overreaction resulting in ARDS and multi organ failure, or in immune dysfunction with infection and sepsis, depending on the particular predisposition.

It is indicated at a certain moment of a clinical development associated with a PMN-dysfunction, to give the organism the supporting treatment to allow to PMN-function returning back to norm. In principle that can be done by pharmacological stimuli of release or activity of pro-inflammatory cytokines (e.g. by neutralizing or receptor blocking antibodies), the induction of apoptosis in activated PMNs, the protection of the effected tissue by anti-oxidants, the prevention of the complement activation, or by the extra-corporeal removal of the activated deregulated PMNs.

Due to the short biologic half life of PMNs, a continuous depletion of activated PMNs is indicated in combination with the removal of pro-inflammatory cytokines, in order to keep the activation potential constantly low.

The most varied and mainly unspecific operating resolutions of the problem exist, based nearly exclusively on in-vitro-experiments, with the goal to terminate the action of different complement factors. They cannot be tested under in vivo conditions because of the expected side effects (e.g. WO-A-98/34959). The inclusion of PMNs in the conceptual formulation and method of resolution did not take place.

An unspecific complement inactivation was introduced in ex-vivo-procedures to prevent complement activation on artificial extra-corporeal surfaces (e.g. surface coating). Furthermore, the U.S. Pat. No. 5,853,722 discloses the selective removal of activated complement factors by using specific anti-C5 antibodies as most preferred, especially highly affine antibodies against all components of the complement system are generated now.

The demonstrated functional cascade preferentially serves the elimination of intruded bacterial. Once there is a discrepancy in number and/or virulence of the intruded bacteria and the elimination capacity of the immune system (e.g. in post traumatic immune deficiency), an overreacting activation can be seen. It is followed by massive release of “shock mediators” (interleukins, platelet activating factor (PAF), but also oxygen radicals, prostaglandins and its metabolites), which result in further inhibition of the elimination capacity. In addition, CD14 negative cells (e.g. endothelia cells) become activated because the soluble CD14 (sCD14) in the blood plasma captures LPS, which makes it more easy to bind on these cells and trigger the release of additional shock mediators, leading into a vicious circle. Because the shock mediators function selectively but not specifically, more functional restriction can be seen in different cells and organs (PMNs, lymphocytes, blood clotting system, circulation, and complement system). The whole organism is attacked by the inflammation reaction which preludes the shock genesis leading to irreversible organ damage, circulatory collapse and death. Different strategies of therapy were examined to interrupt this chain reaction. The cascade interruption with antibodies blocking the LPS binding on proteins (LBP, sCD14), on receptors (CD14), on released cytokines or on cytokine receptors, or with antagonists which block the functional part of the receptors, demonstrated in animal sepsis models; indeed impressive results, but until now, no successful clinical prevention or therapy study is presented.

Schefold et al. (Immunadsorption of endotoxin, IL6 and C5a reverses immunparalysis and improves organ function in patients with severe sepsis and septic shock, Shock 2007; 28:419-426) removed endotoxin, IL-6 land C5a by immune adsorption. As a result, the monocyte function in immune paralyzed patients could be improved significantly. The PMN function was not included. Any influence of immune adsorption on the number of leucocytes could not be proven.

The efforts to remove the pro-inflammatory cytokines by means of extra-corporeal blood cleansing in order to bring the misbalanced immune system back to norm reaction, also underlines that the extra-corporeal intervention of the immunologic dysfunction could be one way of therapeutic interference of these frequently life threatening situations. Hirano et al. (Modulation of Polymorphonuclear Leukocyte Apoptosis in the Critically Ill by Removal of Cytokines with Continuous Hemodiafiltration, Blood Purif 2004; 22:188-197) could prove, that continuous hemodiafiltration causes a significant reduction of cytokine (IL-6), connected with an increased apoptosis rate in PMNs.

Whereas a great number of reports exist regarding the extra-corporeal depletion of mediators by unspecific (dialysis, hemodiafiltration, and adsorption on membranes and albumin) or specific procedures (immune adsorption) and how they influence clinical and immune biologic parameters, the reports about therapeutic cell depletion are without any exception related to unspecific adsorption procedures on polymer surfaces.

The ambitious expectations could not be fulfilled. More and more have been realized that the deregulation of the immune system is not only the result of the LPS action on cells and tissue, but a cause-effect-relation of a much more complex nature. The development of the multi organ failure is a very dynamic process of primarily different genesis, in which different mediators and cell population cause very different reactions within a short period of time. This primarily life maintaining function leads quickly to multi organ failure due to false regulation.

The specific separation of activated PMNs for research purposes is realized by magnetic methods by means of monoclonal antibodies against CD11b. That is not applicable for use in humans and/or in continuous procedures.

The purpose of the invention is the development of a procedure, which can be used in form of a medical device for the extra-corporeal treatment of diseases like:
Acute Respiratory Distress Syndrome (ARDS) and multiple organ failure in result of multiple lesions, severe surgery trauma, lung injury, etc.
Hemolytic Uremic Syndrom (HUS)
Ischemic or post-ischemic conditions after injuries, inflammations, burnings, infarcts, operations, radiologic or toxic action on e.g. gastro-intestine, kidney, pancreas, liver, heart, brain, lung, skin and other organs and tissues.
Post-ischemic conditions after severe loss of blood.
Sepsis and septic shock
All further pathogenic conditions which go along with the PMN-dysfunction.

According to a preferred embodiment, the combination of continuous depletion of activated PMNs and simultaneously or in close time allow for the reduction of pro-inflammatory PMN activating mediators. The inclusion of complement depletion, preferentially C5a, is always indicated when organ damage is expected. Furthermore, the inactivation of activated complement within the medical device improves its biocompatibility.

Although the complete PMN fraction can in principal be extra-corporeally removed by known filtration procedures, especially because at the beginning of the treatment of life-threatening situations like septic shock nearly all PMNs are activated, the targeted depletion of activated PMNs according to the invention is of advantage for the return of the PMNs to physiologic apoptosis rate and rebalancing of the immune system to norm regulation. The systematic removal of all PMNs would have fatal consequences. Together with the activated PMNs or close in time, the PMN activating pro-inflammatoric cytokines like TNFα, IL-1β, IL-6, IL-8, GM-CSF, and interferon-gamma, preferably IL-6, are removed from the blood.

### BRIEF SUMMARY OF THE INVENTION

A filter system for the continuous removal of activated PMNs from blood is disclosed herein. The filter system comprises molecules that identify and bind activated PMNs, molecules that identify and bind PMN-activating mediators, molecules that identify and bind C3a and/or C5a, biocompatible surfaces (carrier) on which those molecules are covalently or adoptively bound, a housing to carry the activated carrier, and valves and tubes.

According to a preferred embodiment, the molecules that identify and bind activated PMNs are polypeptides containing an amino acid sequence of natural or artificial origin, which identify and bind on specific structures of the surface of activated PMNs.

According to another preferred embodiment, the molecules that identify and bind PMN-activating mediators are polypeptides containing an amino acid sequence of natural or artificial origin, which identify and bind PMN-activating mediators.

According to another preferred embodiment, the molecules that identify and bind C3a and/or C5a are polypeptides containing an amino acid sequence of natural or artificial origin, which identify and bind C3a and/or C5a.

According to another preferred embodiment, the polypeptides can be antibodies of any origin.

According to another preferred embodiment, the antibodies are monoclonal and/or polyclonal.

According to another preferred embodiment, the polyclonal antibodies are of avian origin.

According to another preferred embodiment, the mediators are TNFα, IL-1β, IL-6, IL-8, GM-CSF, and interferon-gamma.

According to another preferred embodiment, IL-6 is identified and bound.

According to another preferred embodiment, parts of CD11b/CD18 are identified and bound.

According to another preferred embodiment, a biocompatible carrier of all shapes can be used, but preferably particle.

According to another preferred embodiment, a particle with a diameter between 10-1000 μm are used.

According to another preferred embodiment, the housing is formed in a way which allows the detachment of mediators and cells within or outside of the device for the purpose of regeneration.

According to another preferred embodiment, the filter system is part of a extra-corporeal circuit by means of tubes, valves, and pumps.

According to another preferred embodiment, at least two units are used, so that a continuous blood treatment is possible.

According to another preferred embodiment, the filter system is used in treatment of pathologic situations in individuals which are combined with a dysfunction of PMNs.

According to another preferred embodiment, the filter system is used in treatment of the Acute Respiratory Distress Syndrome (ARDS), multi-organ failure, Hemolytic Uremic Syndrom (HUS), Ischemic or post-ischemic conditions after injuries, inflammations, burnings, infarcts, operations, radiologic or toxic action on e.g. gastro-intestine, kidney, pancreas, liver, heart, brain, lung, skin and other organs and tissues, post-ischemic conditions after severe loss of blood, and sepsis and/or septic shock.

According to another preferred embodiment, the filter system is used in treatment of diseases in which activated complement has a prominent role in the pathogenesis of organ damages like brain trauma, heart infarct or cerebral malaria.

Also disclosed is a procedure for manufacturing of the filter system.

### DETAILED DESCRIPTION OF THE INVENTION

Antibodies are used which preferably recognize activated PMNs as well as the PMN activating cytokines. For the PMN binding, antibodies are preferably used which bind on extra-cellular regions of the CD11b, CD18, or other regions of the CR3 (CD11b/CD18, Mac-1). According to the invention peptides also can be used which represent parts of the antigen binding segment of the specific antibody, or molecules, or sub-units of the specific CD11b ligands (iC3b Fibrinogen, Factor X, FcR II, FcR III, uPAR, CD14, ICAM-1, ICAM-2, ICAM-4, Heparin, Haptoglobin, Kininogen, CD23, Neutrophil inhibitory factor from Ancylostoma C, Filamentous hemagglutinin form Bordetella P, gp63 from Leishmenia D, WI-1 antigen from Blastomyces D).

The immune adsorber comprises as solid support the common membrans, fibers, whole fibers, or particles from organic or synthetic biocompatible polymers, e.g. made from polystyrene, carbohydrates like e.g. derivates from cellulose or agarose, or acrylate, whereat the specific ligands (antibodies or peptides) are covalently bound or them or via spacer or linker.

The manufacturing of the immune adsorber is carried out based on principally known methods, by covalent or adsorbing coupling of antibodies which are directed against CD11b, C5a and IL-6 and if necessary further PMN activating mediators on solid support of organic origin or synthetic polymers.

The specific antibodies are generated based on known immunization procedures with the appropriate antigen, preferentially in small mammals like mice, rats, or rabbits or in birds as e.g. chicken.

Antibodies of any origin can be used. Preferred are monoclonal antibodies or polyclonal antibodies, especially favored are avian antibodies of the type IgY. According to the invention, it comprises antibodies directed against CD11b, C5a and IL-6.

One embodiment of the invention is the use of the adsorber as device to remove activated PMNs, IL-6, complement factor C5a, and when indicated, further mediators from blood depending on the patient's situation.

In a preferred embodiment, the adsorbers are used for the therapy of diseases which are accompanied by PMN dysfunction syndrome. But also therapeutic blood treatment is possible in cases where activated complement plays a prominent pathogenetic role in organ damage, like e.g. brain trauma, heart infarct, or cerebral malaria. Although antibodies against most of the substances are available, which can be coupled with known methods on the different carrier, preferably monoclonal and/or avian antibodies are used. In contrast to mammalian antibodies, avian antibodies do not activate the complement system. Because the activating quality is located in the Fc-part of the mammalian antibodies, principally papain cleaved Fab-fragments can be used or humanized antibodies are applied.

Immobilized avian antibodies have no unspecific affect at all on the defense system of humans. Birds, preferably chicken, are immunized according common procedures with or without the use of adjutants. The specific immune globulins are excreted with the egg yolk, and can be isolated by known methods. They are coupled with familiar procedures on the Fc-part, on micro-particles, or membranes.

With the adsorption system for the extra-corporeal cell depletion for the first time ever a selective system can be provided, which can be used patient specific and whereby false regulation of the immune system can be resolved. Due to the combination with the depletion of pro-inflammatory PMNs activating cytokines, the PMN-dysfunction syndrome is simultaneously treated (return to norm apoptosis and phagocytosis rate), as well as the systemic effect of an excessive pro-inflammatory reaction situation. The possibility to regenerate the device makes the continuous removal of great cell numbers cost-effective.

1. A filter system for the continuous removal of activated Polymorphonuclear leukocytes from blood, the filter comprising:
a. a plurality of molecules that identify and bind activated PMNs;
b. a plurality of molecules that identify and bind PMN-activating mediators;
c. a plurality of molecules that identify and bind at least one member of the group consisting of C3a, and C5a;
d. a plurality of biocompatible surfaces on which the plurality of molecules that identify and bind the activated PMNs, the plurality of molecules that identify and bind the PMN-activating mediators, and the plurality of molecules that identify and bind at least 1 member of the group consisting of C3a and C5a are covalently or adoptively bound, the plurality of biocompatible surfaces comprising a plurality of activated carriers;
e. a housing carrying the plurality of activated carriers; and
f. a plurality of valves and tubes connected to the housing.

2. The filter system according to claim 1, wherein the plurality of molecules that identify and bind activated PMNs comprise polypeptides containing an amino acid sequence of natural or artificial origin, wherein the polypeptides identify and bind on specific structures of a surface of activated PMNs.

3. The filter system according to claim 1, wherein the plurality of molecules that identify and bind the PMN-activating mediators comprise polypeptides containing an amino acid sequence of natural or artificial origin, wherein the polypeptides identify and bind the PMN-activating mediators.

4. The filter system according to claim 1, wherein the plurality of molecules that identify and bind comprise polypeptides containing an amino acid sequence of natural or artificial origin, wherein the polypeptides identify and bind.

5. The filter system according to claim 2, wherein the polypeptides are antibodies of any origin.

6. The filter system according to claim 5, wherein the antibodies are monoclonal, polyclonal, or monoclonal and polyclonal.

7. The filter system according to claim 5, wherein the antibodies are polyclonal antibodies of avian origin.

8. The filter system according to claim 3, wherein the PMN-activating mediators are selected from the group consisting of TNFα, IL-1α, IL-6, IL-8, GM-CSF, and interferon-gamma.

9. The filter system according claim 8, wherein the PMN-activating mediator is identified and bound.

10. The filter system according claim 2, wherein the PMNs comprise a human cell surface receptor CD11b/CD18, and wherein parts of the human cell surface receptor CD11b/CD18 are identified and bound.

11. The filter system according to claim 1, wherein the plurality of activated carriers has a particle shape.

12. The filter system according to claim 11, wherein the particle has a diameter between 10-1000 μm.

13. The filter system according to claim 1, wherein the housing is formed in a way which allows detachment of the PMN-activating mediators within or outside of the housing.

14. The filter system according to claim 1, wherein the filter system is part of a extra-corporeal circuit.

15. A method comprising treating blood using at least two embodiments of the filter system of claim 1 such that a continuous blood treatment is effected.

16. A method comprising treating a pathologic situation in a human in need of treatment for a dysfunction of PMNs comprising treating blood of the human being with the filter system of claim 1.

17. The method of claim 16, wherein the human is in need of treatment for a disorder selected from the group consisting of comprising treating Acute Respiratory Distress Syndrome (ARDS), multi-organ failure, Hemolytic Uremic Syndrome (HUS), Ischemic or post-ischemic conditions after injuries, inflammations, burnings, infarcts, operations, radiologic or toxic action on gastro-intestine, kidney, pancreas, liver, heart, brain, lung, skin, human organs and tissues, post-ischemic conditions after severe loss of blood, sepsis, septic shock and sepsis.

18. A method comprising treating diseases in which an activated complement has a prominent role in a pathogenesis of organ damages by filtering blood with the filter system of claim 1, wherein the disease is selected from the group consisting of brain trauma, heart infarct or cerebral malaria.

19. A method for production of a filter system comprising manufacturing the filter system of claim 1.
